(19) United States
(12) Patent Application Publication
(10) Pub. No.: US 2009/0010975 A1
(43) Pub. Date: Jan. 8, 2009

(54) Non-transparent composition for film

(75) Inventor: Sumihiro Shiraishi, Yamatokoriyama-shi (JP)

Correspondence Address: FITCH, EVEN, TABIN & FLANNERY, P. O. BOX 18415, WASHINGTON, DC, 20036, US

(73) Assignee: QUALICAPS CO., LTD., Yamatokoriyama-shi (JP)

(21) Appl. No.: 12/213,586

(22) Filed: Jun. 20, 2008

Related U.S. Application Data
(60) Provisional application No. 60/945,190, filed on Jun. 20, 2007.

(51) Int. Cl. A61K 9/70; A61K 9/00; A23L 1/00; A61K 9/48

(52) U.S. Cl. 424/401; 424/443; 424/451; 426/89

(57) ABSTRACT

The present invention provides a non-transparent film composition that has light-shielding properties and can thereby maintain the stability and quality of products, such as formulations, for a long period of time; and formulations using this film composition, and particularly filled capsules. The non-transparent film composition contains a water-soluble metal compound containing at least one metal selected from the group consisting of sodium, potassium, calcium, magnesium, aluminium, manganese, iron, cobalt, nickel, copper, strontium, and barium; and a water-soluble cellulose derivative. The non-transparent film composition is preferably prepared by: spreading an aqueous solution containing a water-soluble cellulose derivative and a water-soluble metal compound containing a monovalent, divalent or trivalent metal into a film- or sheet-like form; and heating the solution at a temperature of 60° C. or higher to obtain the film composition by drying and solidification.

### TECHNICAL FIELD

The present invention relates to a film composition that has a light-shielding effect and can thereby maintain the stability and quality of products, such as formulations, for a long period of time; and formulations using this film composition, and particularly filled capsules.

### BACKGROUND ART

Some components contained in drugs, quasi-drugs, foods, and cosmetics are unstable when exposed to light, oxygen, and water (humidity). To prevent the deterioration of such components due to light and ensure the quality stability, products as mentioned above are generally coated with a film composition having a light-shielding effect.

For example, titanium oxide is often used as a light-shielding agent. However, some pharmaceutical components are unstable in the presence of titanium oxide, or their decomposition is promoted due to radicals generated from titanium oxide by UV irradiation. Therefore, the use of titanium oxide with certain pharmaceutical components is prohibited. When formulations are coated with a film composition containing titanium oxide, film peeling or whitening may be caused by light (UV) irradiation over time. If such phenomena occur, the coating effect and light-shielding effect as well as the appearance are impaired, and components contained inside may be decomposed by light, oxygen, and water, thus resulting in serious problems.

To solve the above problem, a variety of research has been conducted (see, for example, U.S. Pat. No. 6,187,340B1 and AU1430602A). However, none of the known methods is satisfactory, and the development of a novel film composition having a sufficient light-shielding effect has been desired.

In view of the prior art, an object of the present invention is to provide a novel non-transparent film composition having a light-shielding effect, and a method of production thereof. Another object of the invention is to provide a formulation coated with the non-transparent film composition, and a capsule (base material) formed using the non-transparent film composition.

### DISCLOSURE OF THE INVENTION

The present inventor conducted extensive research to achieve the above-mentioned objects. As a result, the inventor found that an aqueous solution containing a water-soluble cellulose derivative, and a monovalent, divalent, or trivalent metal ion such as sodium, potassium, calcium, magnesium, aluminium, manganese, iron, cobalt, nickel, copper, strontium, or barium ion, is transparent in the form of a solution, but becomes non-transparent when the solution heated at a temperature of 60° C. or more. The inventor ascertained that this aqueous solution can be effectively used to produce a non-transparent film having excellent light-shielding properties, and the film thus obtained is useful as a light-shielding film. The invention has been accomplished based on these findings. The invention includes the following embodiments.

### I. Non-Transparent Film Composition

(I-1) A non-transparent film composition comprising a water-soluble cellulose derivative and a water-soluble metal compound containing a monovalent, divalent, or trivalent metal.

(I-2) A non-transparent film composition according to (I-1), wherein the monovalent, divalent, or trivalent metal is at least one metal selected from the group consisting of sodium, potassium, calcium, magnesium, aluminium, manganese, iron, cobalt, nickel, copper, strontium, and barium.

(I-3) A non-transparent film composition according to (I-1) or (I-2), wherein the water-soluble cellulose derivative is at least one member selected from the group consisting of methylcellulose, hydroxypropyl cellulose, and hydroxypropyl methylcellulose.

(I-4) A non-transparent film composition according to any one of (I-1) to (I-3) which contains the water-soluble metal compound in an amount of 0.1 to 100 parts by weight per 100 parts by weight of the water soluble cellulose derivative, expressed on a solvent-free weight basis when using a solvate of the compound.

(I-5) A non-transparent film composition according to any one of (I-1) to (I-4) obtained by: spreading an aqueous solution containing a water-soluble cellulose derivative and a water-soluble metal compound containing a monovalent, divalent or trivalent metal into a film- or sheet-like form; and heating the solution at a temperature of 60° C. or higher to obtain the film composition by drying and solidification.

### II. Method of Producing the Non-Transparent Film Composition

(II-1) A method of producing a non-transparent film composition comprising (1) spreading an aqueous solution into a film- or sheet-like form, the aqueous solution containing a water-soluble cellulose derivative and a water-soluble metal compound containing a monovalent, divalent or trivalent metal, and (2) heating the solution at a temperature of 60° C. or higher to obtain the film composition by drying and solidification.

(II-2) A method according to (II-1), wherein the monovalent, divalent, or trivalent metal is at least one metal selected from the group consisting of sodium, potassium, calcium, magnesium, aluminium, manganese, iron, cobalt, nickel, copper, strontium, and barium.

(II-3) A method according to (II-1) or (II-2), wherein the water-soluble cellulose derivative is at least one member selected from the group consisting of methylcellulose, hydroxypropyl cellulose, and hydroxypropyl methylcellulose.

(II-4) A method according to any one of (II-1) to (II-3), wherein the aqueous solution contains the water-soluble metal compound in an amount of 0.1 to 100 parts by weight per 100 parts by weight of the water soluble cellulose derivative, expressed on a solvent-free weight basis when using a solvate of the compound.

### III. Use of Non-Transparent Film Composition

(III-1) A formulation coated with the non-transparent film composition of any one of (I-1) to (I-5).

(III-2) A formulation according to (III-1), which is a tablet, a granule, or a filled capsule.

(III-3) A capsule comprising the non-transparent film composition of any one of (I-1) to (I-5).

(III-4) A filled capsule comprising the capsule of (III-3) filled with a drug, food, or cosmetic.

### BEST MODE FOR CARRYING OUT THE INVENTION

### I. Non-Transparent Film Composition and Method of Preparation Thereof

A feature of the non-transparent film composition is containing a water-soluble metal compound containing a monovalent, divalent, or trivalent metal, and a water-soluble cellulose derivative.

Examples of the water-soluble cellulose derivative used in the invention include cellulose ethers substituted with at least one alkyl or hydroxyalkyl group. Examples of the “alkyl group” of the alkyl or hydroxyalkyl group include C₁-C₆, and preferably C₁-C₄, straight- or branched-chain lower alkyl groups. Specific examples thereof include methyl, ethyl, butyl, and propyl. Specific examples of the water-soluble cellulose derivative include lower alkyl celluloses such as methyl cellulose and ethyl cellulose; hydroxy lower alkyl celluloses such as hydroxyethyl cellulose and hydroxypropyl cellulose; hydroxyl-lower alkyl-alkyl celluloses such as hydroxyethyl methylcellulose, hydroxyethyl ethylcellulose, and hydroxypropyl methylcellulose; and cellulose-based, water-soluble polymers such as hydroxypropylmethylcellulose phthalate, hydroxypropylmethylcellulose acetate phthalate, and carboxymethylethyl cellulose. Among these, methyl cellulose, hydroxypropyl cellulose and hydroxypropyl methylcellulose are preferable, and hydroxypropyl methylcellulose is particularly preferable.

The water-soluble cellulose derivative used in the invention may be any water-soluble cellulose derivative that does not prevent the solution from having a kinematic viscosity of 40 to 40,000 mm²/s when the solution is formed into a film or a sheet. As long as this requirement is met, a wide variety of commercially available water-soluble cellulose derivatives can be used. Such water-soluble cellulose derivatives can be used singly or in a combination of two or more, as long as the above requirement is satisfied. In general, the ratio (Mw/Mn) of the weight average molecular weight (Mw) to the number average molecular weight (Mn) of a commercially available water-soluble cellulose derivative is in the range of 1.5 to 4. The weight average molecular weight (Mw) and the number average molecular weight (Mn) used to calculate this ratio (Mw/Mn) can be determined by gel chromatography (size-exclusion chromatography). With respect to the principle and method of gel chromatography, reference can be made to, for example, “Size-Exclusion Chromatography” in the Chromatography section of USP30-NF25 (the United States Pharmacopeia/National Formulary).

Examples of the monovalent, divalent, or trivalent metal include monovalent metals such as sodium and potassium; divalent metals such as calcium, magnesium, manganese, iron, cobalt, nickel, copper, strontium, and barium; and trivalent metals such as aluminum and iron.

The water-soluble metal compound is not particularly limited as long as it dissolves in water, an organic solvent, or a mixture thereof to release a monovalent, divalent, or trivalent metal ion as mentioned above. Specific examples of the water-soluble metal compound include water-soluble oxides, hydroxides, inorganic salts, and organic acid salts of various monovalent, divalent, or trivalent metals as mentioned above. The water-soluble metal compound used may be a solvate such as a hydrate, or may be a complex. Such metal compounds can be used singly or in a combination of two or more.

Examples of water-soluble inorganic salts of a monovalent, divalent, or trivalent metal include fluorides, chlorides, bromides, carbonates, hydrogen carbonates, phosphates, hydrogen phosphates, monohydrogen phosphates, dihydrogen phosphates, hydroxides, silicates, sulfates, hydrogen sulfates, nitrates, and like salts of monovalent, divalent, or trivalent metals, as mentioned above. Among these, chlorides, carbonates, phosphates, and sulfates are preferable, and chlorides and sulfates are particularly preferable.

Examples of water-soluble organic acid salts of a monovalent, divalent, or trivalent metal include acetates, citrates, tartrates, pantothenates, gluconates, succinates, glycerophosphates, saccharates, stearates, ascorbates, lactates, and like salts of various monovalent, divalent, or trivalent metals. Lactates and gluconates are preferable. The “saccharic acid” of the saccharates refers to carboxylic acid obtained by formal oxidation of the aldehyde group of aldose.

The non-transparent film composition of the invention essentially contains the aforementioned water-soluble cellulose derivative and water-soluble metal compound, and may optionally contain various additives such as plasticizers, sequestering agents, flavors, and coloring agents.

The plasticizer used herein is not particularly limited. Examples thereof include dioctyl adipate, polyester adipate, epoxidized soybean oil, epoxyhexahydrophthalate diesters, kaolin, triethyl citrate, glycerol, glycerol fatty acid esters, acetyl glycerol fatty acid esters, sesame oil, dimethylpolysiloxane-silicon dioxide mixture, D-sorbitol, medium chain fatty acid triglyceride, corn starch-derived sugar alcohol solutions, triacetin, concentrated glycerol, castor oil, phytosterol, diethyl phthalate, dioctyl phthalate, dibutyl phthalate, butyl phthalyl butyl glycolate, propylene glycol, polyethylene glycol, polyoxyethylene (105) polyoxypropylene (5) glycol, polysorbate 80, polyethylene glycols with an average molecular weight of 1,500, 400, 4,000,600, and 6,000 (PEG 1500, PEG 400, PEG 4000, PEG 600, and PEG 6000), isopropyl myristate, cotton seed oil-soybean oil mixture, glyceryl monostearate, and isopropyl linolate. The average molecular weight of PEG can be determined in accordance with the following testing method specified by the Ministry of Health, Labour and Welfare, as described in “Japanese Pharmacopoeia” and “Japanese Pharmaceutical Excipients”.

### Average Molecular Weight Test

42 g of phthalic anhydride is added to a 1 L light-resistant, ground stopper bottle containing exactly 300 mL of newly distilled pyridine. The bottle is vigorously shaken to dissolve the phthalic anhydride, and the solution is allowed to stand for 16 hours or longer. Exactly 25 mL of the resulting solution is measured out into an about 200-mL pressure-resistant, ground stopper bottle, and a specific amount, i.e., about 0.8 to about 12.5 g, of a PEG sample to be tested is precisely measured out into the bottle. The bottle is tightly sealed, then wrapped with a strong cloth, and immersed into a water bath preheated at 98±2° C. in such a manner that the liquid in the bottle is under the water level of the bath. After the water bath is kept at 98±2° C. for 30 minutes, the bottle is removed from the bath and allowed to cool in air to room temperature. Subsequently, precisely 50 mL of a 0.5 mol/L sodium hydroxide solution is added, and five drops of a solution of phenolphthalein in pyridine (0.01 g/mL) are added. The resulting liquid is titrated with a 0.5 mol/L sodium hydroxide solution. Titration is terminated when the liquid can maintain a pale red color for 15 seconds. A blank test is carried out in the same manner as above.

Average molecular weight=(quantity of sample (g)×4,000)/(a−b)

a: Amount (mL) of the 0.5 mol/L sodium hydroxide solution consumed in the blank test

b: Amount (mL) of the 0.5 mol/L sodium hydroxide solution consumed in the PEG sample test

Examples of sequestering agents that can be used include ethylenediaminetetraacetic acid, acetic acid, boric acid, citric acid, gluconic acid, lactic acid, phosphoric acid, tartaric acid, and salts thereof, metaphosphate, dihydroxyethylglycine, lecithin, β-cyclodextrin, and combinations thereof.

The coloring agent is not particularly limited, but is preferably a pharmaceutically acceptable coloring agent.

A gelling agent may also be used as required. Examples of gelling agents that can be used include carrageenan, tamarind seed polysaccharide, pectin, xanthan gum, locust bean gum, curdlan, gelatin, furcellaran, agar, and gellan gum. Such gelling agents can be used singly or in a combination of two or more. In general, three types of carrageenan—kappa-carrageenan, iota-carrageenan, and lambda-carrageenan—are known. Kappa- and iota-carrageenans with gelling ability can suitably be used in the invention. Pectins can be classified into LM pectin and HM pectin according to the esterification degree. Gellan gums can also be classified into acylated gellan gum (native gellan gum) and deacylated gellan gum, depending on whether they are acylated. All such pectins and gellan gums can be used herein, regardless of the type.

When a gelling agent is used, a gelling aid can also be used according to the type of the gelling agent used. Examples of gelling aids that can be used together with carrageenan are as follows. When kappa-carrageenan is used as a gelling agent, examples of gelling aids that can be used together include compounds capable of donating in water one or more types of metal ions selected from potassium ions, ammonium ions, and calcium ions, such as potassium chloride, ammonium chloride, ammonium acetate, or calcium chloride. When iota-carrageenan is used as a gelling agent, examples of gelling aids that can be used together include compounds capable of donating calcium ions in water, such as calcium chloride. When gellan gum is used as a gelling agent, examples of gelling aids that can be used together include compounds capable of donating in water one or more types of ions selected from sodium ions, potassium ions, calcium ions, and magnesium ions, such as sodium chloride, potassium chloride, calcium chloride, and magnesium sulfate. In addition, citric acid or sodium citrate can also be used as an organic acid or a water-soluble salt thereof.

When hydroxypropyl methylcellulose is used as a water-soluble cellulose derivative, the gelling agent and gelling aid preferably used therewith may be, for example, carrageenan and potassium chloride, respectively.

Although the proportion of the water-soluble cellulose derivative in the non-transparent film composition of the invention is not particularly limited, it is usually 40 to 98 wt. %, preferably 50 to 95 wt. %, more preferably 70 to 95 wt. %, and further preferably 80 to 92 wt. %. The proportion of the water-soluble metal compound is preferably in the range of 0.05 to 150 parts by weight, more preferably from 0.1 to 100 parts by weight, still more preferably from 0.2 to 40 parts by weight, and still further preferably from 1 to 20 parts by weight, per 100 parts by weight of the water-soluble cellulose derivative contained in the non-transparent film composition. When the water-soluble metal compound used is a solvate, the part by weight of the water-soluble metal compound specified above is expressed on a solvent-free weight basis.

When the non-transparent film composition of the invention contains a gelling agent, the content thereof may be in the range of 0.05 to 10 wt. %, preferably 0.1 to 9.5 wt. %, more preferably 0.2 to 9 wt. %, and still more preferably 0.3 to 8 wt. %. When the non-transparent film composition of the invention further contains a gelling aid such as potassium chloride, the content thereof may be in the range of 2.2 wt. % or less, preferably 0.1 to 2.1 wt. %, more preferably 0.2 to 1.9 wt. %, and still more preferably 0.3 to 1.6 wt. %. When the non-transparent film composition of the invention contains a plasticizer, the content thereof may usually be in the range of 15 wt. % or less, preferably 13 wt. % or less, more preferably 11 wt. % or less, and still more preferably 8 wt. % or less. When the non-transparent film composition of the invention contains a coloring agent, the content thereof can be appropriately selected from the range of not more than 15 wt. %, preferably 13 wt. % or less, more preferably 11 wt. % or less, and further preferably 8 wt. % or less, according to the desired color density.

The non-transparent film composition of the invention can be prepared, for example, by dissolving a water-soluble cellulose derivative and a water-soluble metal compound, and optionally various additives, a gelling agent, and a gelling aid in a solvent such as water, spreading the solution into a film- or sheet-like form, and distilling off the solvent at a temperature of 60° C. or higher to obtain a film by drying and solidification. The solvent is not limited to water. For example, an organic solvent such as ethyl alcohol, methyl alcohol or like alcohols, diethyl ether, dimethyl ether, or like ethers, acetone or like ketones, and a mixture thereof can also be used as the solvent. Water, ethyl alcohol, methyl alcohol, and a mixture thereof are preferable.

In the production of the non-transparent film composition, the solution used for forming a film or a sheet contains each component in a proportion that does not prevent the solution from having a kinematic viscosity of 40 to 40,000 mm²/s when the solution is formed into a film or a sheet. More specifically, the solution may contain the water-soluble cellulose derivative in a proportion of 1 to 60% by weight, preferably 5 to 50% by weight, and more preferably 10 to 30% by weight, and may contain the water-soluble metal compound in a proportion of 0.06 to 30% by weight, preferably 0.25 to 20% by weight, and more preferably 0.3 to 10% by weight, although the proportions are not limitative. When the water-soluble metal compound used is a solvate, the proportion of the water-soluble metal compound is expressed on a solvent-free weight basis. The solution preferably contains the water-soluble metal compound in a proportion of 0.05 to 150 parts by weight, more preferably from 0.1 to 100 parts by weight, still more preferably from 0.2 to 40 parts by weight, and still further preferably from 1 to 20 parts by weight (expressed on a solvent-free weight basis when using a solvate of the compound), per 100 parts by weight of the water-soluble cellulose derivative contained in the solution.

It is usually preferable that the kinematic viscosity of the solution when being formed into a film or a sheet be in the range of 40 to 40,000 mm²/s, as mentioned above. The kinematic viscosity is more preferably from 90 to 22,000 mm²/s, still more preferably from 350 to 22,000 mm²/s, and still further preferably from 5,000 to 15,000 mm²/s. The kinematic viscosity specified herein can be determined in accordance with the method described in Experimental Example 2.

The non-transparent film composition can be produced by any method that comprises flow-casting or spreading a solution containing the above-mentioned components into a film- or sheet-like form, and heating the solution to obtain the film composition by drying and solidification, as described above. Any method, such as the solvent casting, casting, calendering, extrusion, T-die molding, and inflation molding, that satisfies this requirement can be used, regardless of the type of method.

More specifically, for example, one production method comprises dissolving a water-soluble metal compound in water heated to about 70° C. to about 80° C.; adding a water-soluble cellulose derivative to the solution to form a dispersion; cooling the dispersion to about 40° C. to about 50° C. to form a gelled solution (preferably having a kinematic viscosity of 40 to 40,000 mm²/s); extending (flow casting) the gelled solution into a film or a sheet on a flat plate; and heating the gelled solution at a temperature of 60° C. or higher to obtain a film composition by drying and solidification. Another production method, which utilizes the water-soluble cellulose derivative's ability to form a gel at a temperature of 60° C. or higher, comprises extending (flow casting) the above-mentioned cooled solution into a film or a sheet on a flat plate heated to 60° C. or higher to gel the solution, and simultaneously dry and solidify the gel at a temperature of 60° C. or higher. When a gelling agent and a gelling aid are used in addition to the water-soluble cellulose derivative, the resulting solution forms a gel when cooled. Therefore, a method comprising extending (flow casting) the solution into a film or a sheet on a cooled flat plate, or extending and then cooling the solution to form a gel, and thereafter drying and solidifying the gel at 60° C. or higher can be used.

The heating temperature used for drying and solidification may usually be 60° C. or higher. The upper limit of the heating temperature is not particularly limited. The heating temperature is usually 60° C. to 150° C., and preferably 60° C. to 100° C.

When the solution is formed into a film or a sheet, the thickness of the non-transparent film composition (film thickness) can be appropriately adjusted. The thickness of the non-transparent film composition is usually 5 μm or more, preferably from 20 to 2,000 μm, and more preferably from 20 to 500 μm.

A feature of the non-transparent film composition of the invention thus obtained is that the composition is non-transparent in the dry state. The non-transparency of the film composition can be evaluated based on the light transmittance of the dry film composition. More specifically, the light transmittance of the dry film composition when irradiated with light is measured as the lightness (L value) using a spectrophotometer in accordance with the method described below, to determine whether the composition is non-transparent. As shown in Experimental Example 1 below, the transparency gradually decreases as the relative value of the lightness (L value) of the film composition decreases to 90 or lower. The composition is non-transparent when the relative value is 70 or lower. Therefore, the relative value of the lightness (L value) of the non-transparent film composition of the invention, as measured under the conditions described below, is 70 or lower, and preferably 65 or lower.

### <Evaluation of the Degree of Non-Transparency>

(1) Each dry film composition to be tested is set on a cell holder and irradiated with light using a halogen lamp (standard illuminant: D₆₅/10). The lightness (L value) of the film is determined using a spectrophotometer (“SE-2000”; a product of Nippon Denshoku Industries Co., Ltd.).

(2) As a control experiment, the lightness (L value) is measured in the same manner as above without setting any dry film composition on the cell holder. Setting the lightness (L value) obtained in the control experiment to 100, the relative value of the lightness (L value) of each film is calculated.

A water-soluble transparent layer or a water-insoluble support layer, preferably a waterproof layer (a water-resistant layer), may be formed on at least one surface of the sheet or film of the non-transparent film composition of the invention.

The water-soluble transparent layer formed on at least one surface of the sheet or film of the non-transparent film composition may be any water-soluble, transparent film or sheet formed by using a water-soluble film base. Examples of the water-soluble film base include known water-soluble film bases such as, for example, cellulose polymers such as methylcellulose, ethylcellulose, methylhydroxyethylcellulose, hydroxypropylcellulose, hydroxypropylmethylcellulose, hydroxypropylmethylcellulose phthalate, hydroxypropylmethylcellulose acetate succinate, and carboxymethylethylcellulose; synthetic polymers such as polyvinylacetal diethylaminoacetate, aminoalkyl methacrylate copolymer E (EUDRAGIT E (trade name); a product of Röhm Pharma), ethyl acrylate-methyl methacrylate copolymer (EUDRAGIT NE (trade name); a product of Röhm Pharma), polyvinyl alcohol, polylactic acid, and polyvinylpyrrolidone; polysaccharides such as pullulan, alginic acid, dextrin, mannitol, chitosan, and hemicellulose; and acrylic acid-based polymers such as methacrylic acid copolymer L (EUDRAGIT L (trade name); a product of Röhm Pharma). Such water-soluble film bases can be used singly or in a combination of two or more. The film or sheet can be formed by any of the above-mentioned methods, such as the solution casting (casting), calendering, extrusion, T-die molding, and inflation molding. More specifically, for example, a method comprising extending a solution containing the water-soluble film base into a film or a sheet on a flat plate, followed by drying and solidification can be used.

The solution used for forming such a water-soluble transparent layer contains the aforementioned water-soluble film base and may further contain various additives (such as plasticizers, sequestering agents, flavors, and coloring agents), a gelling agent, and a gelling aid.

A laminate composed of a film or sheet of the non-transparent composition, and a water-soluble transparent layer formed on at least one surface thereof can be produced by preparing a film or sheet of the non-transparent film composition by the above-mentioned method, placing thereon a water-soluble transparent layer (a water-soluble transparent film or sheet) formed by the aforementioned method, and attaching the transparent layer to the film or sheet (the non-transparent layer). The water-soluble transparent layer may be attached to the film or sheet by, for example, applying an alcohol solution of a water-soluble polymer to the surfaces to be bonded, and bonding the surfaces to each other. Such a laminate can also be produced by applying a solution containing the water-soluble film base to the surface of a film or sheet of the non-transparent film composition so as to extend (flow cast) the solution into a film or a sheet, followed by drying and solidification.

The thickness of the water-soluble transparent layer (water-soluble transparent film or sheet) is not particularly limited, but can be appropriately selected from the range of 5 μm or more, preferably from 20 to 2,000 μm, and more preferably from 20 to 500 μm.

When a water-insoluble support layer is formed on at least one surface of a film or sheet of the non-transparent film composition or a laminate composed of the film or sheet and a water-soluble transparent layer formed thereon, the material for the water-insoluble support layer is not particularly limited, as long as it is water-insoluble, and preferably waterproof or water-resistant. Examples of the material include synthetic resins such as polyethylene, polypropylene, and polyethylene terephthalate; and laminated paper.

The support layer (preferably waterproof layer) may be transparent or of any color, and may have desired letters or designs printed thereon.

Examples of the production method thereof include, but are not limited to, a method in which a film or sheet of a non-transparent composition, or a two-layer film or sheet composed of a water-soluble transparent layer and a water-soluble, non-transparent layer is prepared, and such a film or sheet is placed on a support layer and bonded thereto; a method in which a solution containing the components for forming a non-transparent layer is applied to a surface of the support layer to extend the solution into a film or a sheet, and dried and solidified; and a method in which a solution containing the aforementioned water-soluble cellulose derivative and water-soluble metal compound for forming a water-soluble, non-transparent layer is applied to a surface of the support layer to extend the solution into a film or a sheet and heated at a temperature of 60° C. or higher for drying and solidification, after which a solution containing a film base for forming a water-soluble transparent layer is applied to the water-soluble, non-transparent layer to extend the solution into a film or a sheet, and dried and solidified.

### II. Use of the Non-Transparent Film Composition

### (II-1) Coating of Formulations

The non-transparent film composition of the present invention can be used as a covering material for various formulations. The target formulation of the invention is not limited to pharmaceuticals, but also includes quasi-drugs, foods, and cosmetics.

Although the coating method is not particularly limited, known general methods can usually be used. Examples of usable methods include spray coating, pan coating, roll coating, and the like. Spray coating is preferable. Spray coating comprises, for example, spraying a coating solution containing a water-soluble metal compound and a water-soluble cellulose derivative over the surface of a formulation (using a coating apparatus such as a Hicoater or Flow Coater), and drying.

The dosage form of the target formulation of the invention may be, for example, tablets, pills, powders, granules, fine granules, and filled capsules. Tablets, pills, granules, and filled capsules are preferable. The filled capsule of the invention includes both a filled hard capsule and a filled soft capsule.

The amount of non-transparent film composition used for coating can be appropriately adjusted and determined according to the dosage form. For example, tablets may be coated with the non-transparent film composition in an amount of 3 to 15 wt. % per 100 wt. % of the final tablets. The amount of non-transparent film composition in other dosage forms can also be suitably adjusted based on the amount of the film composition used in tablets.

Various types of formulations coated with the non-transparent film composition of the invention are protected from light by being covered with the non-transparent film composition of the invention. The non-transparent film composition of the invention can prevent light from adversely affecting the contents of formulations, thereby maintaining the stability and quality of the contents for a long period of time.

### (II-2) Capsules and Filled Capsules

The capsule (film) of the present invention may consist of the non-transparent film composition of the invention, or may be produced by covering a known capsule made of, for example, gelatin, a water-soluble cellulose derivative (e.g., HPMC), or pullulan with the non-transparent film composition of the invention.

The capsule of the present invention has a light-shielding effect, and thus can maintain the stability and quality of the contents, such as pharmaceuticals, of the capsule for a long period of time.

An example of the method of producing the latter capsule is described below.

### (i) Preparation of Capsule-Forming Solution (Immersion Fluid)

One example of the method of preparing a capsule-forming solution comprises dissolving a water-soluble metal compound, optionally together with a gelling agent and a gelling aid, in water heated to about 70° C. to about 80° C.; dispersing a water-soluble cellulose derivative in the solution; and cooling the dispersion to the desired immersion fluid temperature (usually 30° C. to 80° C., preferably 40° C. to 60° C., and more preferably 50° C. to 60° C.). Another example of the method comprises dispersing a water-soluble cellulose derivative in hot water of about 70° C. or higher; cooling the dispersion to about 35° C. or lower to dissolve the water-soluble cellulose derivative; heating the solution again to about 35° C. to about 50° C.; adding and dissolving a water-soluble metal compound, optionally together with a gelling agent and a gelling aid; and adjusting the resulting solution to the desired immersion fluid temperature.

### (ii) Preparation of Capsules

A capsule-forming pin is immersed into the capsule-forming solution (immersion fluid), and then removed therefrom. A film of the solution formed on the outer surface of the capsule-forming pin is allowed to cool to form a gel at 35° C. or less. The gel capsule (gel film) formed on the outer surface of the capsule-forming pin is heated at 60° C. or higher, more specifically, usually in the range of 60° C. to 150° C., to dry and solidify the capsule. The dry capsule is released from the capsule-forming pin.

The heating temperature used for drying and solidification is not particularly limited, as long as it is within the range of 60° C. to 150° C., and preferably 60° C. to 100° C., and more preferably 60° C. to 80° C. The heating can usually be performed by sending air of 60° C. or higher.

The capsule thus prepared is cut to a predetermined length, and then provided as a non-transparent hard capsule (film) composed of a body and a cap unengaged or engaged with each other.

Preferably, the capsule of the invention prepared by the above method contains the water-soluble cellulose derivative in an amount of 70 to 98 wt. %, preferably 75 to 95 wt. %, more preferably 80 to 95 wt. %, and even more preferably 80 to 92 wt. %, and contains the water-soluble metal compound in an amount of 0.01 to 40 part by weight, preferably 0.05 to 33 parts by weight, more preferably 0.1 to 25 parts by weight, and further preferably 1 to 25 parts by weight, per 100 parts by weight of the water-soluble cellulose derivative contained in the non-transparent film composition. When the water-soluble metal compound used is a solvate, the part by weight of the water-soluble metal compound specified above is expressed on a solvent-free weight basis.

In addition to the above components, the capsule of the invention preferably contains a gelling agent in an amount of 0.05 to 10 wt. %, preferably 0.1 to 9.5 wt. %, and more preferably 0.2 to 9 wt. %, and even more preferably 0.3 to 8 wt. %. When the capsule contains a gelling aid such as potassium chloride, the content thereof may be in the range of 2.2 wt. % or less, preferably 0.1 to 2.1 wt. %, more preferably 0.2 to 1.9 wt. %, and even more preferably 0.3 to 1.6 wt. %. When the capsule of the invention contains a plasticizer, the content thereof is usually 15 wt. % or less, preferably 13 wt. % or less, more preferably 11 wt. % or less, and further preferably 8 wt. % or less. When the capsule contains a coloring agent, the content thereof is usually appropriately selected from the range of 15 wt. % or less, preferably 13 wt. % or less, more preferably 11 wt. % or less, and further preferably 8 wt. % or less, according to the desired level of coloring.

The capsule of the invention thus constituted has a light-blocking effect, and thus prevents light from adversely affecting the contents of the capsule, thereby maintaining the stability and quality of the contents for a long period of time.

A filled capsule can be produced by filling the capsule of the invention with a drug, a food, a cosmetic, etc.

The substance that can be filled into the capsule of the invention is not particularly limited as long as it does not dissolve or react with the capsule (film) of the invention. Examples of such substances include solid materials such as powders or granules; liquids; and gels. Liquids that can be filled into the capsule include alcohols such as stearyl alcohol, cetanol, polyethylene glycols having an average molecular weight of 600, 800, 1,000, 1,500, 2,000, 3,000, 4,000, 6,000, 8,000 or 20,000 (PEG600, PEG800, PEG1000, PEG1500, PEG2000, PEG3000, PEG4000, PEG6000, PEG8000, PEG20000); oils and fats such as sesame oil, soybean oil, arachis oil, corn oil, hydrogenated oil, paraffin oil, and white beeswax; fatty acids such as stearic acid, palmitic acid, myristic acid, triethyl citrate, triacetone, and medium-chain triglyceride, and derivatives thereof. Such liquids are usually mixed with active ingredients or the main component of a drug, food, or cosmetic, and filled into the capsule of the invention.

The type of drug that can be filled into the capsule of the invention is not particularly limited, and typical examples thereof are oral drugs. Specific examples thereof include, but are not limited to, vitamins, antifebriles, painkillers, antiphlogistics, anti-tumor agents, cardiotonics, anticoagulants, hemostats, osteoclastic inhibitors, vascularization inhibitors, antidepressants, antiulcer drugs such as proton pump inhibitors including benzimidazole derivatives, expectorants/cough suppressants, antiepileptic agents, antiallergic agents, antiarrhythmics, vasodepressors, hypotensive diuretics, diabetic medicine, antituberculous agents, hormone drugs, and antinarcotics.

The type of food that can be filled into the capsule of the invention is not particularly limited. Examples of such foods include, but are not limited to, functional ingredients such as docosahexaenoic acid, eicosapentaenoic acid, α-lipoic acid, royal jelly, isoflavone, agaricus, acerola, aloe, aloe vera, turmeric, L-carnitine, oligosaccharide, cacao, catechin, capsaicin, chamomile, agar, tocopherol, linolenic acid, xylitol, chitosan, GABA, citric acid, chlorella, glucosamine, ginseng, coenzyme Q10, brown sugar, collagen, chondroitin, bracket fungus, squalene, stevia, ceramide, taurine, saponin, lecithin, dextrin, Houttuynia cordata, niacin, Bacillus natto, bittern, lactic acid bacteria, saw palmetto, honey, Coix lacryma-jobi var. ma-yuen, ume extract, pantothenic acid, hyaluronic acid, vitamin A, vitamin K, vitamin C, vitamin D, vitamin B1, vitamin B2, vitamin B6, vitamin B12, quercetin, protein, propolis, mulukhiya, folic acid, lycopene, linoleic acid, rutin, and Ganoderma lucidum.

The filling of such substances into the capsule of the invention can be performed by using a known capsule-filling machine, such as a fully automatic capsule-filling machine and a capsule-filling/sealing machine. An example of the fully automatic capsule-filling machine is Qualicaps's fully automatic capsule filling machine (model name: LIQFIL super 80/150). An example of the capsule-filing/sealing machine is Qualicaps's capsule filling/sealing machine (model name: LIQFIL super FS).

### EXAMPLES

The present invention is described in greater detail below with reference to Experimental Examples and Examples, but the invention is not limited to these Examples.

### Experimental Example 1

Using a spectrophotometer (SE-2000; a product of Nippon Denshoku Industries Co., Ltd.), various films with different degrees of transparency were set on a cell holder, and the lightness (L value) of each film when irradiated with light (D₆₅/10) using a halogen lamp was measured. As a control experiment, no film was set on the cell holder, and the lightness (L value) was similarly measured by directing light in the same manner. Setting the lightness (L value) obtained in the control experiment to 100, the relative value of the lightness (L value) of each film was calculated. Table 1 shows the results of the relative value, and the degree of non-transparency of each film evaluated with the naked eye.

TABLE 1

| Relative | Degree | |
|---|---|---|
| Value of | of Non- | Appearance (Color) of |
| Each Film | Transparency | the Film |
| 95.03 | − | Transparent film |
| 90.54 | − | Transparent film |
| 87.40 | ± | Pale white film |
| 86.70 | ± | Pale white film |
| 65.47 | + | White film |
| 52.45 | ++ | White film |
| 40.63 | ++ | White film |
| 24.62 | ++ | White film |
| 24.04 | ++ | White film |
| 21.57 | ++ | White film |
| 20.83 | ++ | White film |
| 18.92 | ++ | White film |
| 17.01 | ++ | White film |
| 16.33 | ++ | White film |
| 14.84 | ++ | White film |
| 14.02 | ++ | White film |
| 11.91 | ++ | White film |
| 10.62 | ++ | White film |

Degree of non-transparency:
−: completely transparent
±: relatively transparent
+: relatively non-transparent
++: completely non-transparent

These results show that as the transmittance of light of the film decreases, the relative value decreases; hence, there is a positive correlation between the transmittance of light and lightness (relative value). The above results show that the non-transparent films have a relative value of 70 or lower, and preferably 65 or lower, as measured under the above conditions.

### Experimental Example 2

Hydroxypropyl methylcellulose (HPMC) (a product of Shin-Etsu Chemical Co., Ltd.; weight average molecular weight: 60,000; Mw/Mn=1.9 (measured by gel chromatography; the same applies hereinafter)) was measured out into wide-mouthed bottles to the concentrations (3 to 18 wt. %) shown in Table 3, and calcium chloride was added to a final concentration of 2%, after which hot water was added to make a total of 500 g.

After each container was covered with a lid, the mixture was stirred using a stirrer at 350 to 450 revolutions per minute for 10 to 20 minutes, until a homogeneous dispersion was formed. The dispersion was dissolved with stirring in a water bath of 10° C. or lower for 20 to 40 minutes, and the viscosity of the solution was measured. The viscosity was determined at 20±0.1° C. in accordance with the rotational viscometer method using a single cylindrical rotational viscometer (a Brookfield viscometer, LV Model), under the following conditions.

TABLE 2
Operating Conditions

| Viscosity | Cylinder | Revolutions/ | Conversion |
|---|---|---|---|
| (mPa · s) | No. | min. | multiplier |
| 600 to less | 3 | 60 | 20 |
| than 1,400 | | | |
| 1,400 to less | 3 | 12 | 100 |
| than 3,500 | | | |
| 3,500 to less | 4 | 60 | 100 |
| than 9,500 | | | |
| 9,500 to less | 4 | 6 | 1,000 |
| than 99,500 | | | |
| 99,500 or more | 4 | 3 | 2,000 |

### Operation of the Apparatus

After the single cylindrical rotational viscometer was operated and rotated for 2 minutes, the measurement on the viscometer was read, and the viscometer was stopped for 2 minutes. This cycle was repeated, and the average of a total of three measurements (absolute viscosity: mPa·s) was determined.

The obtained solution was degassed under reduced pressure, and allowed to stand at room temperature for 12 hours to obtain clear HPMC gels with different concentrations. The density (mass/volume) of each HPMC gel was measured at 20±0.1° C. Using an apparatus for preparing a thin-layer plate for thin layer chromatography, each HPMC gel was cast on a glass plate to form a thin HPMC gel film, and then dried at 60° C. to 100° C. for 1 hour to form a film with a thickness of about 120 μm.

Table 3 shows the results obtained by evaluating the kinematic viscosity (absolute viscosity/density; unit: mm²/s) and film-forming ability (workability) of each HPMC gel, and the degree of whiteness (non-transparency) of the obtained films.

TABLE 3

| | | Degree of | |
|---|---|---|---|
| HPMC | Kinematic | Whiteness (Non- | Film-Forming |
| Concentration | Viscosity (mm^{2/}s) | transparency and | Ability |
| in HPMC Gel | of HPMC Gel | Hiding Power) | (Workability) |
| 3% | 40 | B | D |
| 4% | 90 | B | B |
| 6% | 350 | A | B |
| 8% | 1000 | A | B |
| 10% | 2500 | A | B |
| 12% | 5000 | A | A |
| 15% | 15000 | A | A |
| 16% | 22000 | A | B |
| 18% | 40000 | A | C |

Degree of whiteness (non-transparency and hiding power)
A: Excellent
B: Good
C: White but has low hiding power
Film-forming ability (workability)
A: Good
B: Capable of forming a film
C: Capable of forming a film, but requires a long degassing time when preparing a gel
D: Difficult to form a film due to low viscosity; but capable of preparing a film by using a mold or a spraying method

The above results show that when dried and solidified at a temperature of 60° C. or higher, and more specifically at 60° C. to 100° C., the HPMC gels containing a water-soluble metal salt containing calcium as a water-soluble metal compound became non-transparent and were produced as films having light-shielding properties. In view of the relationship between the film-forming ability and the degree of whiteness of the obtained films, the results show that the viscosity of the solution when the solution is formed into a film is preferably in the range of 40 to 40,000 mm²/s. The viscosity of the solution is preferably in the range of 90 to 22,000 mm²/s from the viewpoint of the film-forming ability, is preferably 350 to 40,000 mm²/s from the viewpoint of the degree of whiteness, and is particularly preferably 5,000 to 15,000 mm²/s from the viewpoints of the film-forming ability and the degree of whiteness.

### Experimental Example 3

20 g of each of the water-soluble metal compounds shown in Table 4 was dissolved in 830 g of purified water heated to about 80° C., and 150 g of hydroxypropyl methylcellulose (HPMC) (a product of Shin-Etsu Chemical Co., Ltd.; weight average molecular weight: 60,000; Mw/Mn=1.9) was added thereto with stirring to prepare suspensions. The resulting suspensions were dissolved by stirring at a temperature of 50° C. or lower, degassed under reduced pressure, and allowed to stand at room temperature for 12 hours to obtain clear HPMC gels (kinematic viscosity: 8,400 mm²/s). Using an apparatus for preparing a thin-layer plate for thin layer chromatography, each HPMC gel was cast on a glass plate to form a thin HPMC gel film, and then dried at 60° C. to 100° C. for 1 hour to form a film with a thickness of about 120 μm.

Table 4 shows the types of water-soluble metal compounds used, and the results obtained by observing the degree of non-transparency of the obtained films with the naked eye, and the color and hiding power of the films prepared by drying at 60° C. or higher. As comparative samples, films were similarly prepared without using a water-soluble metal compound (Not Used), and the films were evaluated in the same manner.

TABLE 4

| | | | | | | | Color and Hiding |
|---|---|---|---|---|---|---|---|
| | | | | | | | Power of the Film |
| Water-Soluble | Drying Temperature and Degree | | | | | | Produced by |
| Metal | of Non-Transparency | | | | | | Drying at 60° C. or |
| Compound | 25° C. | 50° C. | 60° C. | 70° C. | 80° C. | 100° C. | higher |
| NaCl | − | − | ++ | ++ | ++ | ++ | White and has |
| | | | | | | | hiding power |
| KCl | − | − | ++ | ++ | ++ | ++ | White and has |
| | | | | | | | hiding power |
| CaCl₂ | − | − | ++ | ++ | ++ | ++ | White and has |
| | | | | | | | hiding power |
| (CH₃CHOHCOO)₂Ca•5H₂O | − | − | ++ | ++ | ++ | ++ | White and has |
| | | | | | | | hiding power |
| MgCl₂•6H₂O | − | − | ++ | ++ | ++ | ++ | White and has |
| | | | | | | | hiding power |
| AlCl₃•6H₂O | − | − | ++ | ++ | ++ | ++ | White and has |
| | | | | | | | hiding power |
| MnCl₂•4H₂O | − | − | ++ | ++ | ++ | ++ | White and has |
| | | | | | | | hiding power |
| FeCl₂•4H₂O | − | − | ++ | ++ | ++ | ++ | Dark yellowish- |
| | | | | | | | white and has |
| | | | | | | | hiding power |
| CoCl₂ | − | − | ++ | ++ | ++ | ++ | Dark bluish-white |
| | | | | | | | and has hiding |
| | | | | | | | power |
| NiCl₂ | − | − | ++ | ++ | ++ | ++ | Pale yellowish- |
| | | | | | | | white and has |
| | | | | | | | hiding power |
| CuSO₄•5H₂O | − | − | ++ | ++ | ++ | ++ | Pale bluish-white |
| | | | | | | | and has hiding |
| | | | | | | | power |
| SrCl₂•6H₂O | − | − | ++ | ++ | ++ | ++ | White and has |
| | | | | | | | hiding power |
| BaCl₂•2H₂O | − | − | ++ | ++ | ++ | ++ | White and has |
| | | | | | | | hiding power |
| Not Used | − | − | − | − | − | − | Colorless, |
| | | | | | | | transparent and |
| | | | | | | | no hiding power |

Degree of non-transparency
−: completely transparent
±: relatively transparent
+: relatively non-transparent
++: completely non-transparent

These results show that when dried and solidified at a temperature of at least 60° C. or higher, and more specifically at 60° C. to 100° C., the HPMC gels containing as a water-soluble metal compound a water-soluble metal salt containing sodium, potassium, calcium, magnesium, aluminum, manganese, iron, cobalt, nickel, copper, strontium, or barium became non-transparent and were produced as films having light-shielding properties. In contrast, when dried and solidified at 50° C., all the HPMC gels containing as a water-soluble metal compound a water-soluble metal salt containing sodium, potassium, calcium, magnesium, aluminium, manganese, iron, cobalt, nickel, copper, strontium, or barium became completely transparent, and non-transparent films were not formed.

### Experimental Example 4

To achieve the proportions shown in Table 5, various amounts of magnesium chloride hexahydrate were dissolved in purified water heated to about 80° C., and hydroxypropyl methylcellulose (HPMC) (a product of Shin-Etsu Chemical Co., Ltd.; weight average molecular weight: 60,000; Mw/Mn=1.9) was added thereto with stirring to prepare suspensions. The resulting suspensions were dissolved with stirring at a temperature of 50° C. or lower and then degassed under reduced pressure, after which the solutions were allowed to stand at room temperature for 12 hours to obtain clear HPMC gels (kinematic viscosity: 8,000 mm²/s). Table 5 shows the weight ratio of magnesium chloride (calculated as the anhydride) to HPMC used to prepare each HPMC gel. Each HPMC gel was cast on a glass plate using an apparatus for preparing a thin-layer plate for thin layer chromatography to form a thin HPMC gel film, and then dried at 90° C. for 1 hour to form a film with a thickness of about 150 μm.

The strength of the obtained films was evaluated in accordance with the following method.

### Evaluation of Film Strength

Each film was cut into a rectangular strip (5×2 cm). After one end of the strip was folded to meet the opposite end, the strip was opened to its original state. The state of the strip of the film after being open was evaluated according to the following criteria:

(1) A: the film is completely restored to its original state
(2) B: the film is curled
(3) C: the film has a crease remaining
(4) D: the film is broken

Table 5 shows the results of observing the degree of non-transparency and appearance of the obtained films with the naked eye, and the film strength.

TABLE 5

| | Degree of | | |
|---|---|---|---|
| Gel Composition | Non- | Appearance | |
| (Weight Ratio) | Transparency | (Color) of the | Film |
| HPMC:MgCl₂ | of the Film | Film | Strength |
| 100:1.0 | + | Pale white | A |
| 100:4.69 | ++ | White | A |
| 100:6.10 | ++ | White | A |
| 100:10 | ++ | White | A |
| 100:13 | ++ | White | A |
| 100:14.06 | ++ | White | A |
| 100:23.44 | ++ | White | A |
| 100:30 | ++ | White | A |
| 100:37.51 | ++ | White | A |
| 100:50 | ++ | White | A |
| 100:80 | ++ | White | B |
| 100:100 | + | Pale white | C |

Degree of non-transparency
−: completely transparent
±: relatively transparent
+: relatively non-transparent
++: completely non-transparent

The results show that non-transparent white films with high hiding power can be prepared by adding 1 to 100 parts by weight of a water-soluble metal salt (magnesium chloride hexahydrate) to 100 parts by weight of HPMC.

### Experimental Example 5

Hard capsules of the following Formulation were prepared according to the Preparation Method described below, using various types of water-soluble metal salts (Table 4) used in Experimental Example 3 as the water-soluble metal compound. In the Formulation, the gelling agent and gelling aid are carrageenan and potassium chloride, respectively; and D-sorbitol was used as a plasticizer.

Formulation

| | |
|---|---|
| Hydroxypropyl methylcellulose | 85.6% |
| Water-soluble metal salt (see Table 4) | 8.6% |
| Gelling agent | 0.4% |
| Gelling aid | 0.4% |
| Water | 5% |

### Preparation Method

(1) Each of the various types of water-soluble metal salts, and a gelling aid (potassium chloride) are added to purified water of about 80° C. and dissolved. A gelling agent (carrageenan) is added and dissolved therein with stirring. Subsequently, hydroxypropyl methylcellulose (HPMC) is added with stirring and dispersed therein. The dispersion is cooled to 50° C. with stirring to dissolve HPMC. The resulting solution is heated to 55° C. with stirring, and a plasticizer (D-sorbitol) is added to give a capsule base aqueous solution (immersion fluid).
(2) A capsule-forming pin (pin) is immersed in the obtained immersion fluid.
(3) The pin is removed from the immersion fluid, and the immersion fluid on the surface of the pin is allowed to form a gel at 35° C. or below.
(4) The immersion fluid on the surface of the pin is dried at 70° C. to form a film, and the film in the form of a capsule is released from the pin.
(5) The capsule released from the pin is cut to a predetermined size.

### INDUSTRIAL APPLICABILITY

The non-transparent film composition of the present invention, which is non-transparent and has hiding power, can be effectively used as a light-shielding material, and preferably used as a light-shielding film for formulations (e.g., tablets, pills, granules, filled capsules). Various types of formulations coated with the non-transparent film composition of the invention are protected from light by being covered with the non-transparent film composition of the invention. The non-transparent film composition of the invention can prevent light from adversely affecting the contents of formulations, thereby maintaining the stability and quality of the contents for a long period of time. The capsule (film) of the invention formed using the non-transparent film composition of the invention can prevent light from adversely affecting the contents of the capsule, thereby maintaining the stability and quality of the contents for a long period of time.

1. A non-transparent film composition comprising a water soluble metal compound containing a monovalent, divalent, or trivalent metal, and a water-soluble cellulose derivative.

2. The non-transparent film composition according to claim 1, wherein the monovalent, divalent, or trivalent metal is at least one metal selected from the group consisting of sodium, potassium, calcium, magnesium, aluminium, manganese, iron, cobalt, nickel, copper, strontium, and barium.

3. The non-transparent film composition according to claim 1, wherein the water-soluble cellulose derivative is at least one member selected from the group consisting of methylcellulose, hydroxypropyl cellulose, and hydroxypropyl methylcellulose.

4. The non-transparent film composition according to claim 1 which contains the water-soluble metal compound in an amount of 0.1 to 100 parts by weight per 100 parts by weight of the water-soluble cellulose derivative, expressed on a solvent-free weight basis when using a solvate of the compound.

5. The non-transparent film composition according to claim 1 obtained by: spreading an aqueous solution into a film- or sheet-like form, the aqueous solution containing a water-soluble cellulose derivative and a water-soluble metal compound containing a monovalent, divalent or trivalent metal; and heating the solution at a temperature of 60° C. or higher to obtain the film composition by drying and solidification.

6. A formulation coated with the non-transparent film composition of any one of claims 1 to 5.

7. The formulation according to claim 6, which is a tablet, a granule, or a filled capsule.

8. A capsule comprising the non-transparent film composition of any one of claims 1 to 5.

9. A filled capsule comprising the capsule of claim 8 filled with a drug, food, or cosmetic.

10. A method of producing a non-transparent film composition, comprising (1) spreading an aqueous solution into a film- or sheet-like form, the aqueous solution containing a water-soluble metal compound containing a monovalent, divalent or trivalent metal, and a water-soluble cellulose derivative, and (2) heating the solution at a temperature of 60° C. or higher to obtain the film composition by drying and solidification.

11. The method according to claim 10, wherein the monovalent, divalent, or trivalent metal is at least one metal selected from the group consisting of sodium, potassium, calcium, magnesium, aluminium, manganese, iron, cobalt, nickel, copper, strontium, and barium.

12. The method according to claim 10, wherein the water-soluble cellulose derivative is at least one member selected from the group consisting of methylcellulose, hydroxypropyl cellulose, and hydroxypropyl methylcellulose.

13. The method according to claim 10 wherein the aqueous solution contains the water-soluble metal compound in an amount of 0.1 to 100 parts by weight per 100 parts by weight of the water soluble cellulose derivative, expressed on a solvent-free weight basis when using a solvate of the compound.
